(19) United States
(12) Patent Application Publication
(10) Pub. No.: US 2012/0289960 A1
(43) Pub. Date: Nov. 15, 2012

(54) METHOD AND DEVICE FOR USE OF A SMART SKULL PIN

(76) Inventor: Charles E. Dinkler, II, Dayton, OH (US)

(21) Appl. No.: 13/522,381

(22) PCT Filed: Jan. 14, 2011
(86) PCT No.: PCT/US11/21243

§ 371 Date: Jul. 16, 2012

Related U.S. Application Data
(60) Provisional application No. 61/336,028, filed on Jan. 15, 2010.

(51) Int. Cl. A61B 17/00

(52) U.S. Cl. 606/59

(57) ABSTRACT

A skull pin, a method to assemble the skull pin, and a method to operate the skull pin contemplate a skull pin having a housing defining a recess; a force sensing component, a battery, an electrical component, and a skull pin tip received within the recess, wherein the force sensing component and the battery are electrically connected to the electrical component; the skull pin tip fitting over the force sensing component, wherein the force sensing component provides an output related to the level of force applied to the skull pin tip; and wherein the skull pin tip is secured to the housing by a bearing. The method for assembling the skull pin may include providing a housing with a battery, a force sensing component and an electrical component, wherein the force sensing component and the battery are electrically connected to the electrical component; positioning a skull pin tip within the housing to fit over the force sensing component; and securing the skull pin tip with a bearing, wherein the bearing is pressed over the skull pin tip and into the housing to assemble the skull pin. The method for operating a skull pin contemplates providing the skull pin with a force sensing component and a piezo-electric transducer; securing the skull pin within a head fixation device; applying the skull pin to the head of a patient; and providing an audible alarm with the piezo-electric transducer when a predetermined level of force is sensed.

The present application generally relates to a skull pin and a method for use of a skull pin. The present application more particularly relates to a method and apparatus for assembling and operating a skull pin.

A conventional skull pin is used in a surgical head fixation device to support the head of a patient during neurosurgical and cervical spine procedures. Head fixation devices work like a clamp, or vise, to immobilize the head of a patient having suffered trauma. The head fixation device is generally positioned alongside different areas of the patient's head with skull pins that are directed into the skull at various angles. The skull pins hold the patient's head in a rigid position. During a surgical procedure, the head of the patient may be covered with sterile drapes, while the skull pins need to remain in their position to prevent the patient's head from motion. Certain surgical procedures make it challenging and difficult for the patient's head to be rigidly fixed or for the head fixation device to hold the head under various loads. The head fixation device may also be unable to support the patient's head due to ineffective design or other possible defects. If the patient's head disconnects from the head fixation device, the patient's skin will be lacerated. Furthermore, the slipping of the patient's head will occur under the sterile drape and may not be discovered until the patient is removed from the head fixation device.

Therefore, it is desirable to have a skull pin that rigidly supports the head of a patient while in the head fixation device during neurosurgical and other related procedures that allows feedback of the impending slippages to the surgeon to avoid lacerations of the skin or other possible problems during the surgical procedure. It is also important for a skull pin to communicate the possibility of a drop in force or load to the surgeon by emitting an audible alarm, otherwise it may go undetected while the patient is covered by surgical drapes.

The present application relates to a skull pin. The skull pin includes a housing defining a recess; a force sensing component, a battery, an electrical component, and a skull pin tip received within the recess, wherein the force sensing component and the battery are electrically connected to the electrical component; the skull pin tip fitting over the force sensing component, wherein the force sensing component provides an output related to the level of force applied to the skull pin tip; and wherein the skull pin tip is secured to the housing by a bearing.

The present application also relates to a method for assembling a skull pin. The method includes providing a housing with a battery, a force sensing component and an electrical component, wherein the force sensing component and the battery are electrically connected to the electrical component; positioning a skull pin tip within the housing to fit over the force sensing component; and securing the skull pin tip with a bearing, wherein the bearing is pressed over the skull pin tip and into the housing to assemble the skull pin.

The present application further relates to a method for operating a skull pin. The method includes providing the skull pin with a force sensing component and a piezo-electric transducer; securing the skull pin within a head fixation device; applying the skull pin to the head of a patient; and providing an audible alarm with the piezo-electric transducer when a predetermined level of force is sensed.

These and other features, aspects, and advantages of the present application will become better understood with reference to the following description, appended claims, and accompanying drawings.

The present disclosure will become more fully understood from the following detailed description, taken in conjunction with the accompanying figures, wherein like reference numerals refer to like elements, in which:

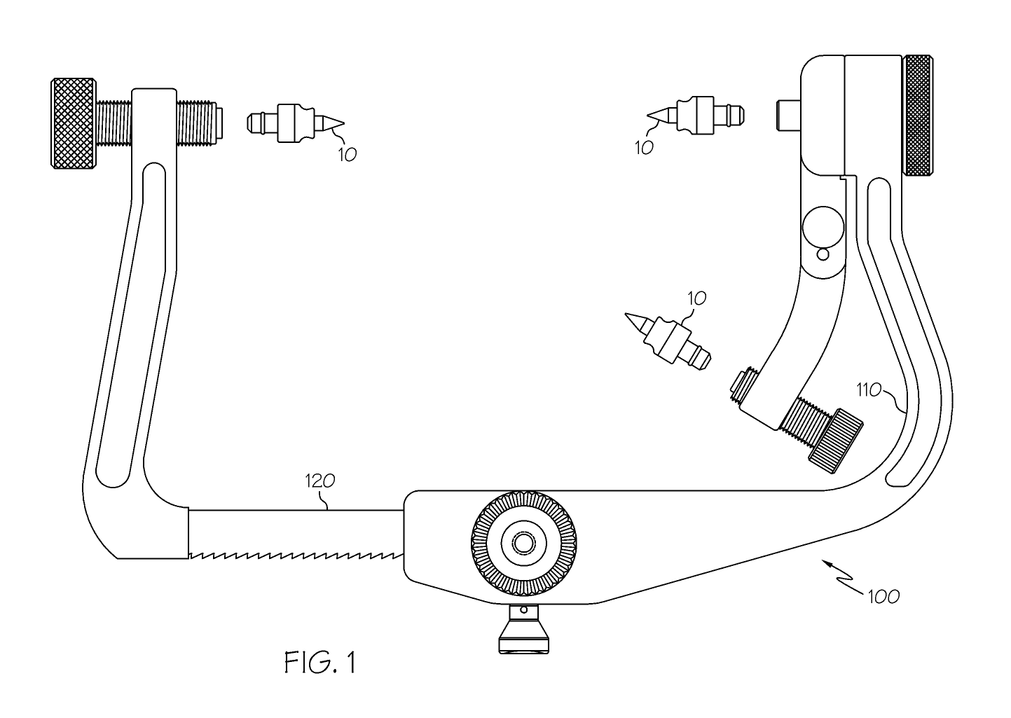

FIG. 1 is a schematic view of a head fixation device, illustrating the placement of skull pins;

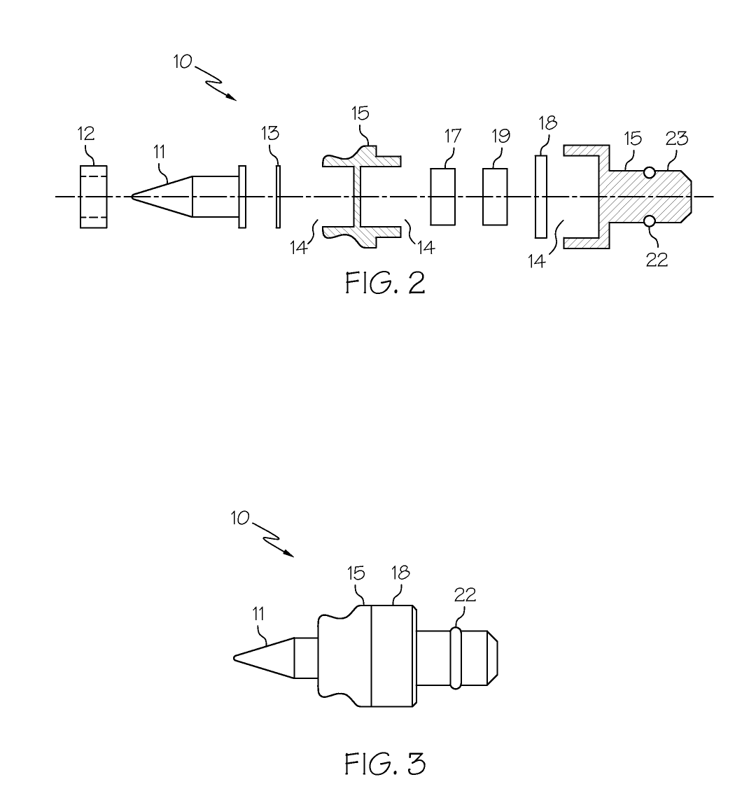

FIG. 2 is an exploded view of a skull pin, illustrating the skull pin and a method for assembling a skull pin, according to an embodiment of the present disclosure;

FIG. 3 is a side view of a skull pin assembled according to an embodiment of the present disclosure; and

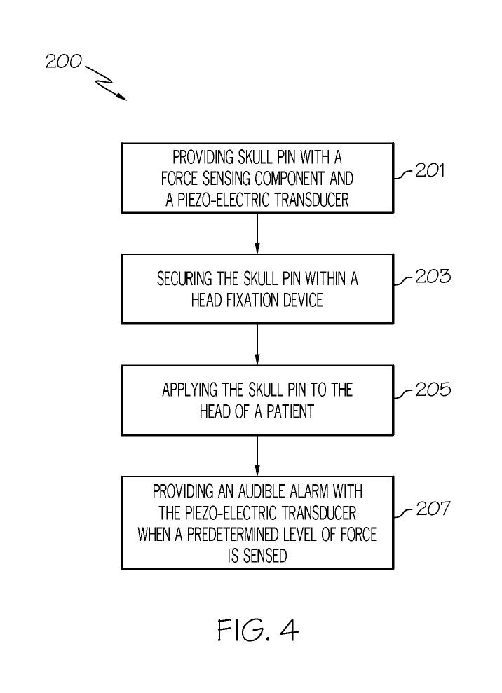

FIG. 4 is a flowchart of an embodiment of a method for operating a skull pin.

Before turning to the figures, which illustrate several embodiments in detail, it should be understood that the application is not limited to the details or methodology set forth in the description or illustrated in the figures. It should also be understood that the terminology used herein is for the purpose of description only and should not be regarded as limiting.

FIG. 1 is a schematic view of a head fixation device, illustrating the placement of skull pins. The head fixation device 100 works like a clamp, or a vise, to keep the head of the patient steady while a surgery is performed. The head fixation device 100 may have a rocker arm 110 containing two skull pins 10 and on the opposing side is a mechanism 120 to engage and advance a third skull pin 10 into the head of a patient.

FIG. 2 is an exploded view of a skull pin, illustrating the skull pin itself and a method for assembling a skull pin, according to an embodiment of the present disclosure. As illustrated, the skull pin 10 has a housing 15 that defines a recess 14. A force sensing component 13, a battery 18, an electrical component 17, and a skull pin tip 11 are received within the recess 14. The force sensing component 13 and the battery 18 are electrically connected to the electrical component 17. The skull pin tip 11 fits over the force sensing component 13, wherein the force sensing component 13 provides an output related to the level of force applied to the skull pin tip 11, and the skull pin tip 11 is secured to the housing 15 by a bearing 12. In other embodiments, the bearing 12 may be omitted. The housing 15 may include a first and a second housing. The first and second housing may be attached together by an interference fit. The skull pin tip 11 may have a point for engaging the skull of a patient and may also be generally conical in shape. The second housing may include an external shaft 23 for engaging a head fixation device. The external shaft 23 may further include a seal 22 that may assist in keeping the skull pin 10 from detaching from the head fixation device 100. The skull pin 10 may also comprise a piezo-electric transducer 19, which is responsive to the electrical component 17, for providing an audible alarm when a predetermined level of force is sensed by the force sensing component 13. In one embodiment, the skull pin tip 11 is smooth. In other embodiments, the skull pin tip may be threaded or splined. The skull pin tip 11 may be fashioned from stainless steel, titanium or combinations thereof. In other embodiments, the skull pin tip may be fashioned from non-conducting materials such as ceramic, plastic, or other suitable materials.

According to FIG. 2, a method for assembling a skull pin is also illustrated. The housing 15 is provided with a battery 18, a force sensing component 13 and an electrical component 17, where the force sensing component 13 and the battery 18 are electrically connected to the electrical component 17. The skull pin tip 11 is positioned within the housing 15 to fit over the force sensing component 13. The skull pin tip 11 is then secured with a bearing 12, where the bearing 12 is pressed over the skull pin tip 11 and into the housing 15 to assemble the skull pin 10. In other embodiments, the bearing 12 may be omitted. The housing 15 may comprise a first and a second housing secured by an interference fit. The housing 15 may include a first bore for receiving the force sensing component 13 and the skull pin tip 11. The housing 15 may include a second bore for receiving the electrical component 17 and the battery 18. The skull pin may also comprise a piezo-electric transducer 19 in the housing 15, which is responsive to the electrical component 17, for providing an audible alarm when a predetermined level of force is sensed by the force sensing component 13. The force sensing component 13 may be wired to the electrical component 17 through an opening in the housing 15. In one embodiment, the electrical component 17 is electrically connected to the battery 18 and the piezo-electric transducer by wiring throughout the skull pin 10. In another embodiment, an insulated wire from the electrical component 17 to the piezo-electric transducer 19 to the battery 18 may be used to connect these components. In another embodiment, an insulated compartment may be used with electrodes to connect the electrical component 17 with the piezo-electric transducer 19 and the battery 18.

FIG. 3 is a side view of a skull pin, assembled according to an embodiment of the present disclosure. FIG. 3 shows the skull pin tip 11, the housing 15, the battery 18 fitted together with a seal 22. The seal may be an o-ring or other type of sealing mechanism. The seal 22 may prevent the skull pin 10 from detaching from the head fixation device 100.

FIG. 4 is a flowchart of an embodiment of a method for operating a skull pin. This method 200 contemplates providing 201 the skull pin 10 with a force sensing component 13 and a piezo-electric transducer 19. The skull pin 10 is secured 203 to a head fixation device 100. The skull pin 10 is then applied 205 to the head of a patient. When a predetermined level of force is applied to the head of the patient 207, the piezo-electric transducer 19 then provides an audible alarm. It will be appreciated that other signaling devices, both audible and visual, may be used in lieu of the piezo-electric transducer 19. For example, a light indicator may be used, or another form of audible signaling device may be incorporated into the skull pin 10.

1. A skull pin comprising:
a housing defining a recess;
a force sensing component, a battery, an electrical component, and a skull pin tip received within the recess, wherein the force sensing component and the battery are electrically connected to the electrical component;
the skull pin tip fitting over the force sensing component, wherein the force sensing component provides an output related to the level of force applied to the skull pin tip; and
wherein the skull pin tip is secured to the housing by a bearing.

2. The skull pin of claim 1, wherein the housing comprises a first and a second housing.

3. The skull pin of claim 1, wherein the first housing is attached to the second housing by an interference fit.

4. The skull pin of claim 1, wherein the skull pin tip has a point for engaging the skull of a patient.

5. The skull pin of claim 1, wherein the skull pin tip is generally conical in shape.

6. The skull pin of claim 1, further comprising a piezo-electric transducer, responsive to the electrical component, for providing an audible alarm when a predetermined level of force is sensed by the force sensing component.

7. A method for assembling a skull pin comprising:
providing a housing with a battery, a force sensing component and an electrical component, wherein the force sensing component and the battery are electrically connected to the electrical component;
positioning a skull pin tip within the housing to fit over the force sensing component; and
securing the skull pin tip with a bearing, wherein the bearing is pressed over the skull pin tip and into the housing to assemble the skull pin.

8. The method of claim 7, wherein the housing comprises a first and a second housing.

9. The method of claim 7, further comprising securing the first housing to the second housing by interference fit.

10. The method of claim 7, wherein the housing includes a first bore for receiving the force sensing component and the skull pin tip.

11. The method of claim 7, wherein the housing includes a second bore for receiving the electrical component and the battery.

12. The method of claim 7, further comprising providing a piezo-electric transducer, responsive to the electrical component, in the housing for providing an audible alarm when a predetermined level of force is sensed by the force sensing component.

13. A method for operating a skull pin comprising:
providing the skull pin with a force sensing component and a piezo-electric transducer;
securing the skull pin within a head fixation device;
applying the skull pin to the head of a patient; and
providing an audible alarm with the piezo-electric transducer when a predetermined level of force is sensed.
